(19) United States
(12) Patent Application Publication
(10) Pub. No.: US 2006/0095011 A1
(43) Pub. Date: May 4, 2006

(54) Waste containment system

(76) Inventor: Alan Aronie, Littleton, MA (US)

Correspondence Address: STEUBING MCGUINNESS & MANARAS LLP, 125 NAGOG PARK, ACTON, MA, 01720, US

(21) Appl. No.: 11/145,401

(22) Filed: Jun. 3, 2005

Related U.S. Application Data
(60) Provisional application No. 60/623,348, filed on Oct. 29, 2004.

(51) Int. Cl. A61F 5/44

(52) U.S. Cl. 604/348; 604/355

(57) ABSTRACT

A waste containment system includes a positionable orifice, removably adjusted to a waist strap to permit individual adjustment of the orifice position depending on user physical characteristics. A waste container includes a collection bag, made of a flexible non-allergenic material that is removably coupled to the orifice. With such an arrangement, the container may be easily replaced when it is full. The container advantageously includes a flap, which extends through the orifice and helps to secure the collection bag to the orifice, while cushioning the user from the container. A compressible seat with a reservoir may advantageously be coupled to the system to accommodate the collection bag when a user is sitting or lying down, minimize discomfort caused to the user in such situations. The system may easily be modified to incorporate existing waste disposal devices, such as diapers, if desired.

### RELATED INVENTIONS

This application claims priority under 35 U.S.C. §1.119(e) to provisional application Ser. No. 60/623,348, entitled Apparatus and Method for Feces Containment and Removal, filed on Oct. 29, 2004.

### FIELD OF THE INVENTION

This invention relates generally to the field of waste containment and more particularly to a system for feces capture and containment.

### BACKGROUND OF THE INVENTION

Many individuals lack the ability to voluntarily retain the passage of urine and excrement. For those individuals, absorbent diapers are generally used to capture the waste for sanitary disposal. The diapers typically are formed from an absorbent material which is positioned against a wearer such that bodily exudates are caught by the article. The waste may then be disposed along with the diaper.

The absorbent cores of diapers, though helpful capturing liquid exudates, are not as effective at capturing solid waste such as feces. Because feces do not generally pass into the absorbent material, they tend to either remain in the diaper, undesirably contacting the wearers' skin and causing irritation and discomfort. When the wearer of a diaper is a child this is generally not a large issue, as children's diapers are relatively frequently changed, and the child may express its discomfort to a caregiver before actual irritation begins.

However, there are other diaper users that are either unable to discern the discomfort, unable to communicate the discomfort, or users that feel a social stigma with dealing with the discomfort at the time that it occurs. Such users must suffer the discomfort, irritation and potential embarrassment of unabsorbed waste in the diaper. If the unabsorbed feces should escape from of the diaper, as often occurs, the diaper wearer, clothing, bedding or other materials near the wearer may become soiled. Such a situation may pose severe health risks to the user. It would be desirable to identify an improved fecal containment system that overcomes the above identified problems.

### SUMMARY OF THE INVENTION

According to one aspect of the invention, a waste disposal system includes a positionable orifice and a disposable container, removably coupled to the positionable orifice. During operation the positionable orifice is positioned and secured using positioning means in alignment with an anal canal of a user such that fecal matter extruded through the anal canal passes through the orifice and into the disposable container. In one embodiment the orifice is made of rubber molded for user comfort. The disposable container is made of a flexible material and includes a collection bag, a neck coupled to the collection bag and a flap coupled to the neck. The neck of the container is extended through the positionable orifice such that the collection bag is on one side of the orifice and the flap lies on the other side of the orifice. At least one face of the flap is formed of a comfortable, advantageously absorbent material. The holding flap may be folded perpendicular to the opening of the orifice to lie flush against the buttocks of the wearer. The positioning means includes a strapping system which allows the positioning of the orifice to be tailored according to the size and shape of the wearer of the system. The waste disposal system may advantageously include a semi-rigid seat, mountable on the positioning means, and a diaper disposed within the semi-rigid seat for capturing liquid wastes. With such an arrangement, a comfortable, sanitary and discrete waste containment system is provided.

### BRIEF DESCRIPTION OF THE DRAWINGS

FIG. 1 is a diagram illustrating several components of the waste containment system of the present invention;

FIGS. 2A-2C illustrate several views of the orifice and positioning straps of the waste containment system of FIG. 1;

FIGS. 3A-3E are views of the two embodiments of a waste container of the present invention, one of which includes a check valve for inhibiting outflow of extrudate from the container;

FIG. 4 is a diagram illustrating the incorporation of the waste container of FIG. 3A or 3D into the positioning system 20 of FIG. 1;

FIG. 5 is a diagram illustrating the incorporation of the waste container of FIG. 3A or 3D into the waste containment system of FIG. 1;

FIG. 6 illustrates one embodiment of a spacer seat which may be coupled to the waste containment components of FIG. 1 to assist in a restriction of movement of the container of FIG. 3A or 3D;

FIG. 7 is a diagram illustrating the incorporation of the spacer seat of FIG. 5 into the waste containment system of FIG. 4;

FIG. 8A illustrates an exemplary diaper which may be included in the waste containment system of the present invention;

FIG. 8B is a diagram illustrating the incorporation of the diaper of FIG. 7A into the waste containment system of FIG. 6; and

FIGS. 9A-8C illustrate the waste disposal system of the FIG. 5 as worn by an individual

### DETAILED DESCRIPTION

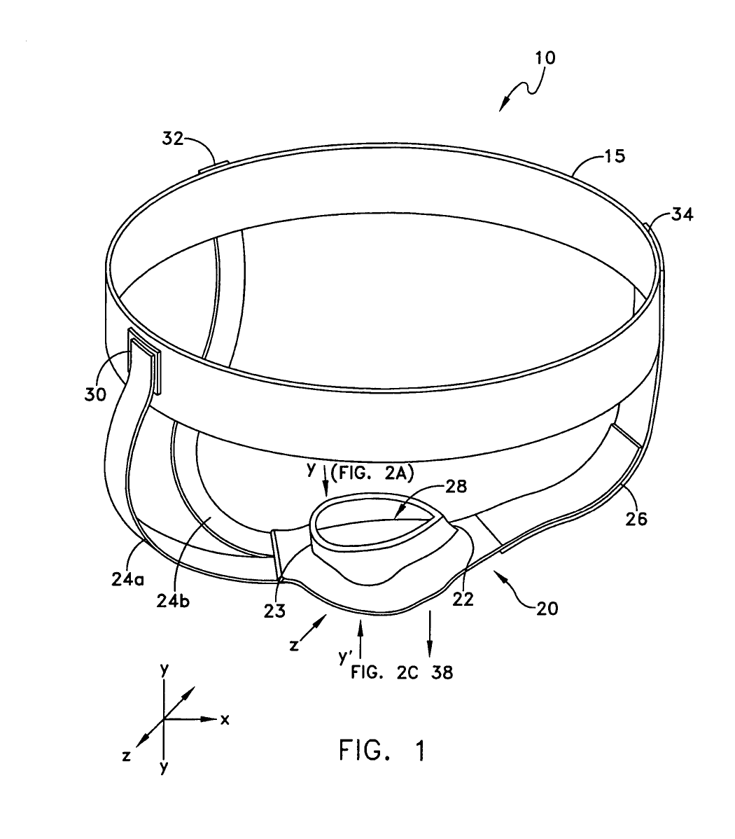

Referring now to FIG. 1, several components of a waste containment system 10 of the present invention include a waist strap 15 and a strapping system 20. The strapping system 20 includes an integrated orifice 22 and a set of front (24a, 24b) and rear (26) straps. In use the straps contact an individual user wearing the containment system, and thus should be selected from a material which is both washable and relatively comfortable. For example, in one embodiment the front straps 24a, 24b may comprise a 1 cm in diameter cylindrical braided rope of nylon, and the rear strap may be made of a 2.5 cm wide nylon web strap. In an alternate embodiment, one or more of the straps may be covered with a removable and/or pad made from cotton or the like, to minimize chafing to the user of the system. Other modifications which are made to the straps to increase comfort and/or cleanliness are within the scope of the present invention and the present invention is not limited to the use of any particular material for the straps.

The embodiment of FIG. 1 illustrates a pair of front straps 24a and 24b and a single back strap. One function of the straps is for positioning and securing the orifice 22 in a desired alignment with an anus of the user, and the tri-strap positioning arrangement shown has several advantages. The two front straps 24a and 24b are spaced to accommodate a user's sexual organs, and therefore permits the device to be secured without interfering with urine flow or causing undue discomfort. The single back strap will naturally fall between the user's buttocks, thereby assisting in the centering orifice at the anus. However, although the three strap configuration is shown in FIG. 1, the present invention is not limited to this particular embodiment, and other embodiments which may include differently placed strap arrangements may be substituted herein as equivalents.

Whatever strap arrangement is selected, at least a subset of the straps may be removably coupled to the waist strap 15 using fastening means 30, 32, 34. The fastening means may be any mechanism that enables attachment and adjustment of the straps at the waist. With such an arrangement, the strapping system may be adjusted to accommodate a variety of bodily configurations. One simple method of providing both adjustment and fastening the straps may be with the use of Velcro hook and loop closures. Alternative methods which include adjustable buckles, snaps or buttons may alternatively be used. In addition, although the strap system of FIG. 1 is shown to have all three straps being removably fastened to the waist strap, it is appreciated that modifications to the present design may include systems where one or more of the straps are fixedly attached to the waist strap, with alternative means for adjusting the length of the strap.

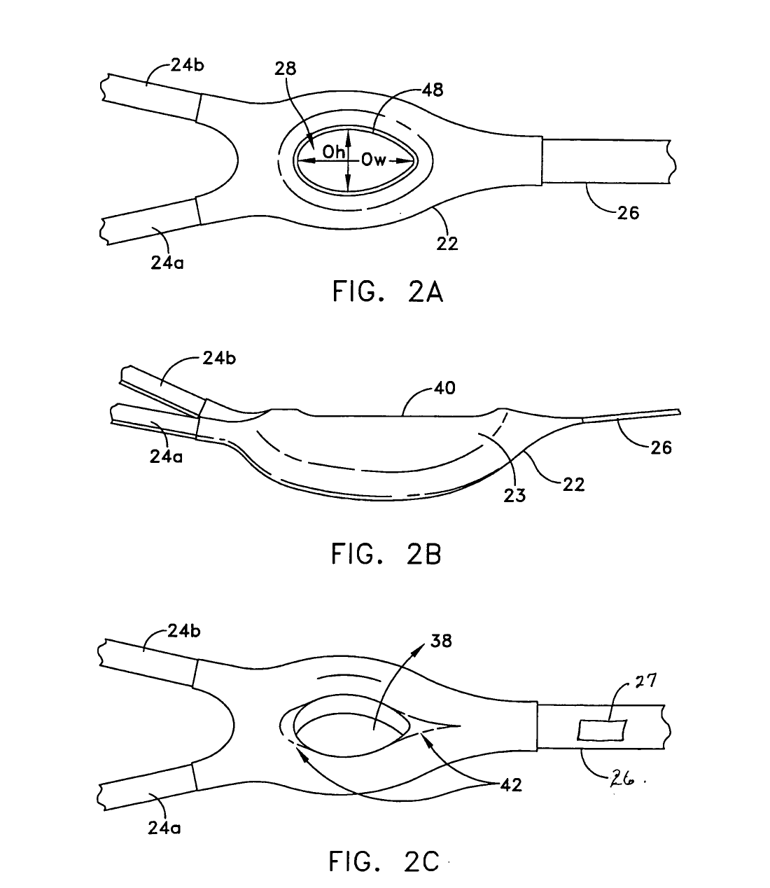

Referring now to FIGS. 2A-2C, several exploded views orifice 22 are shown. In general, because the orifice is to reside against an individual's anus, the material of the orifice is selected from a variety of flexible and easily molded elastomers or polymers. For example, the orifice may be formed from a soft rubber, silicone, polyurethane or other similar material. The orifice may be molded for each individual, but generally comprises a neck 23 which extends from the anus towards the exterior of the buttocks. The orifice includes an inlet 28, defined by a lip 40 which, during operation surrounds the users' anus. The size and shape of the inlet is selected in accordance with a size of an anal opening. In general the inlet is elliptically shaped, and in one embodiment has a width Ow is equal to 7 cm, and the height of the inlet Oh of 4 cm. It is appreciated that the dimensions and shape of the inlet are a matter of design choice, based on the particular physical characteristics and measurements of an individual user. A narrower inlet may be used for smaller individuals, such as youths. The selection of inlet size may also vary based on the fecal matter consistency; for example, it may be desirous to have a larger inlet for individuals with looser stools, to capture a wider dissipation of waste. Other factors may also be considered in the design of the orifice, and the present invention is not limited to any particular design methodology; rather, any orifice that is used to capture fecal matter may be substituted readily herein.

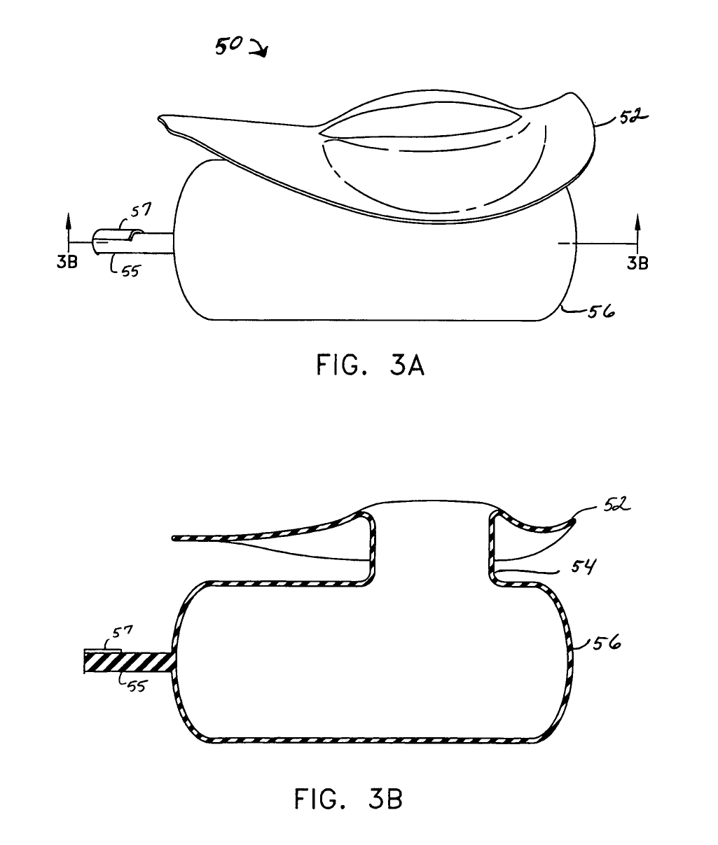

The shape of the orifice and the flexible nature of the material helps to seal the lips 40 of the orifice against the skin of the buttocks that surrounds the anus. During operation pressure from the extrusion of the feces further urges the lips of the flexible material of the orifice against the skin of the user. The pressure forces the orifice further open and minimizes leakage of feces by directing the feces through the orifice into the container. When the pressure is removed, the lips resume their original shape to ensure containment of the feces a coupled container (as will be described in FIGS. 3A and 3B).

FIG. 2A illustrate a top down view of the orifice, taken along the Y axis in FIG. 1, and illustrates generally the lip 40 which defines the inlet 28. FIG. 2B is a side view of the orifice, taken along the Z axis of FIG. 1, and illustrates generally how the orifice is molded in shape to the general shape of a human buttock. FIG. 2C is a bottom view of the orifice, and illustrates the outlet path 38 of the orifice, through which fecal matter passes during use. As shown in FIG. 2C, the strap 26 may include a fastening means 27, which can be used as described later herein to further secure the fecal container to the positioning straps.

FIGS. 3A and 3B are different views of a first embodiment of a container 50 that is used to collect the fecal matter that is extruded through the outlet 38. The container 50 includes a collection bag 56 coupled to a neck 54. The collection bag 56 is preferably made of a lightweight, resilient plastic which is resilient enough to hold fecal waste, even in the event of pressure. An exemplary material is nitrile, such as that used in surgical glove. Nitrile is soft, latex-free, powder-free, and non-sticky, with a low chemical content which reduces risk of allergic reactions. The possibility of contact with human skin makes the nitrile a preferred material due to its non-allergenic properties, however other materials may be readily substituted provided they posses a like resilience.

The collection bag 56 is shown in FIG. 3A to be generally cylindrical in shape, although this shape is provided merely for illustration purposes and does not limit the design. The size and shape of the collection bag is a matter of design choice. In general the bag 56 should be large enough to accommodate fecal matter that may be extruded by an individual. It is appreciated that the amount and consistency of fecal matter is largely based on the diet of the individual user and thus can vary user to user and day to day. In several studies it has been shown that a maximum range of fecal matter extruded in a day may be in the range of 500 grams, and the collection bag may be sized to accommodate this amount. However, this value is provided for purposes of illustration only; the capacity of the collection bag should generally be sufficient enough to accommodate fecal matter without bursting or leakage. In the exemplary embodiment of FIG. 3A, the collection bag has a length of 9 inches and a width of 7 inches. In one embodiment the collection bag may be formed of two portions and sealed around the edges. Alternatively the collection bag may be molded into a unitary piece. In one embodiment of the invention the collection bag includes a finger shaped protrusion 55. The protrusion 55 may include a fastening means 57 which may be used to assist with fastening the container to the strapping system to minimize movement of the container when in use.

The container 50 includes an integrated neck 54. Prior to use of the waste containment system, the neck 54 is threaded through the outlet of the orifice towards the inlet, and thus the height 1 of the neck is matched to the height of the neck 23 of the orifice. During operation, fecal matter is extruded through the neck 54 into the collection bag 56. The neck 54 is roughly matched in diameter to the dimensions of the inlet of the orifice, although it may be advantageously smaller to reduce the possibility of back flow of fecal matter into the inlet. For example, in the exemplary embodiment described above, with the inlet Ow of 7 cm and Oh of 4 cm, the diameter of the neck may be 2.125 cm. In one embodiment, a check valve such as check valve 45 in FIG. 3C may be incorporated into the neck to prevent back flow of the fecal matter out of the inlet of the orifice, however it will be appreciated that the inherent flexibility and resilience of the nitrile material may provide functionality similar to the check valve by expanding to allow the fecal matter to exit the outlet, and then returning to its original shape to prevent the back flow. An embodiment 49 of a container including the check valve 45 is illustrated in FIG. 3D. As shown in FIG. 3C, the check valve includes a flattened tube portion 43. The opening 41 of the flattened tube portion 43 extends into the into the opening of the orifice as shown in FIGS. 3D and 3E. Once the extrudate is pushed through the opening 47 of the check valve and passes through the opening 41 of the flattened tube 43, the check valve inhibits backflow of the extrudate through the orifice.

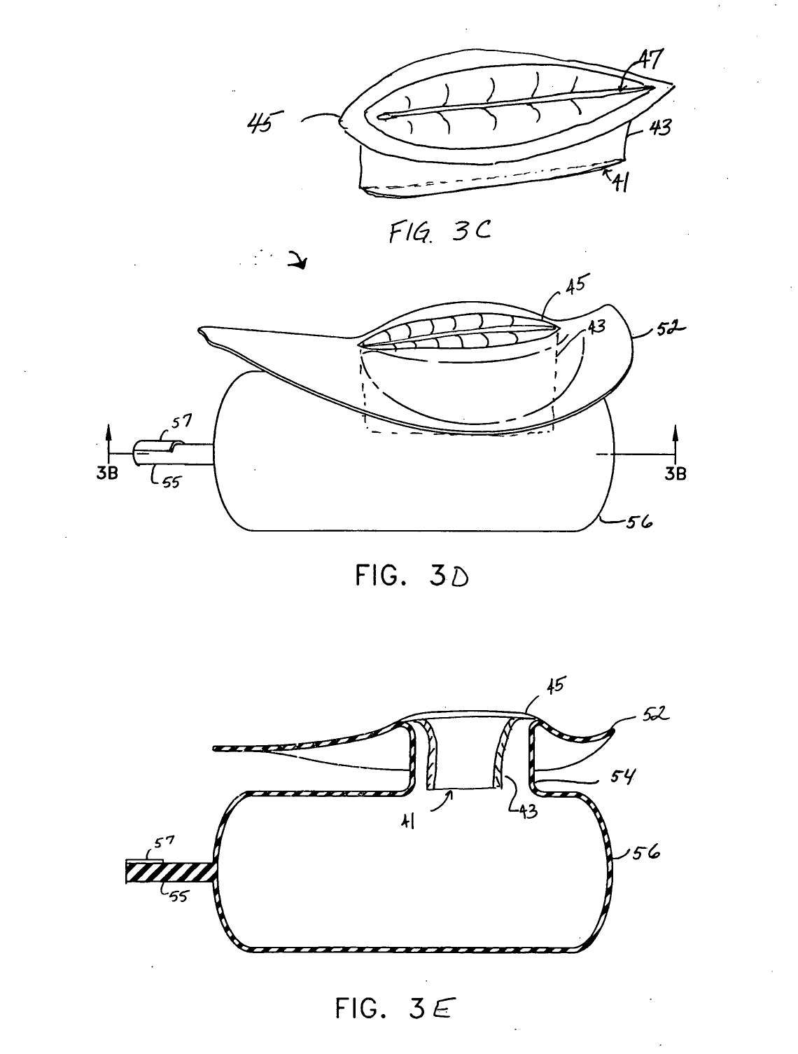

Referring again to FIGS. 3A and 3B, according to one embodiment of the invention, the container 50 includes a holding flap 52, coupled to the inlet opening 53 of the neck 54. The holding flap 52 serves the purpose of securing the collection bag on the orifice. To ensure that the collection bag is secured to the orifice, the flap is sized in one embodiment to generally match the shape of the orifice, and extends outward from the neck and can be secured by an individual's weight during use. For example, FIG. 4 illustrates how the container is positioned in the strapping system, with the holding flap matched in general shape and size to the orifice 22.

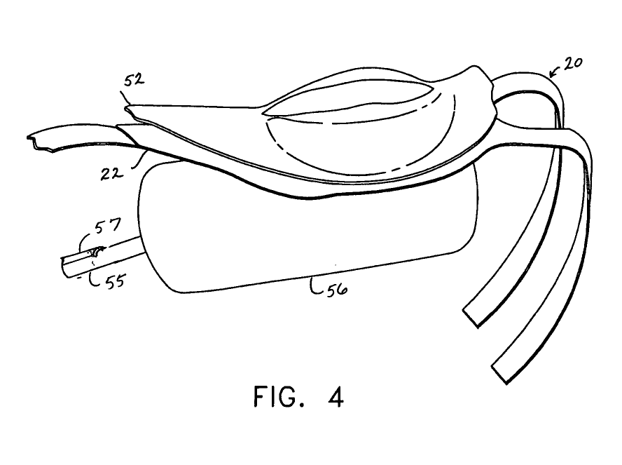

In an embodiment where the flap may be integrated into a wipe 59 as shown in FIG. 5. The wipe serves a dual purpose of shielding the user from the strapping system and collection bag, as well as being usable to wipe the user's anus upon removal of the collection bag. The material of the wipe is therefore generally selected to provide comfort to the user during use. For example, the material may be absorbent material which assists in the capture of urine or sweat from the user.

FIG. 5 also illustrates the placement of the container in the strapping system of FIG. 1. As shown in FIG. 4, the neck of the container 50 extends through the orifice 22, with the collection bag at the outlet of the orifice, and the flap 52 (here shown contained in wipe 59) at the inlet of the orifice. During operation, the flap/wipe combination is folded back in perpendicular alignment with the opening of the orifice, and will lie over the strap 26 and bag 56, thereby shielding the user from contact with these elements of the waste containment system. The finger 55 extends back towards strap 26 and the securing means 57 on the finger 55 attaches to securing means 27 on strap 26 to further secure the container in the strapping system to prevent sway and droop of the bag during operation.

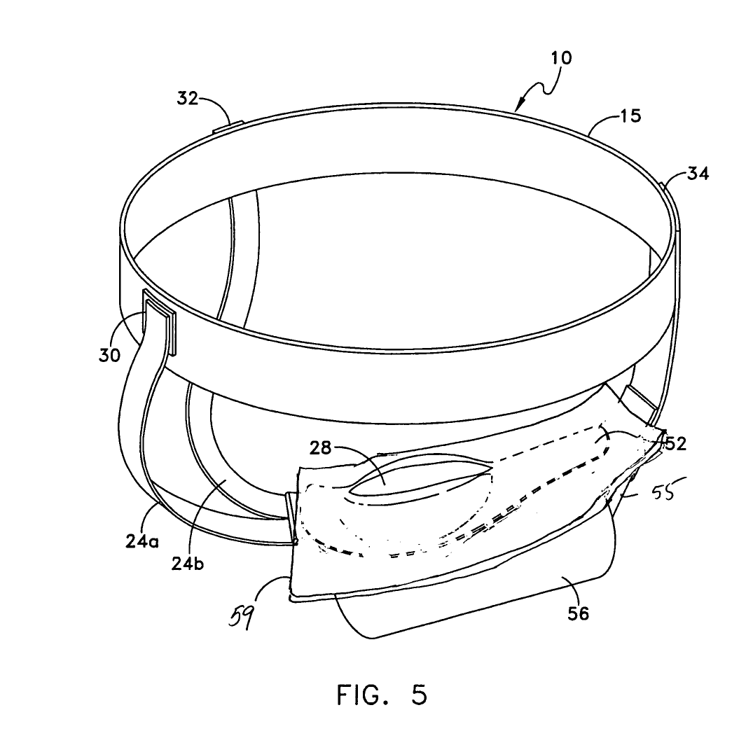

The elements shown in the waste containment system of FIG. 5 are useful in those situations where a user is not likely to lie or sit on the bag 56. However, there are many situations where the free movement of the bag 56 outside the waste system is not desirous. For this reason, according to another aspect of the invention, a seat 60 such as that shown in FIG. 6 may be coupled to the system of FIG. 5. The seat 60 in one embodiment is comprised of lightweight, compressible foam, and includes several passages 64a, 64b and 66 which are positioned to accept the straps of the strapping system 20. The seat 60 also includes a reservoir 65, within which the collection bag resides when the waste disposal system and seat are coupled to the user. The seat 60 may take any form, but is generally sized according to the physical shape of the user, with the reservoir sized to accommodate a filled collection bag. When the seat is included in the waste collection system, the user need not worry about placing pressure on the collected waste, and any associated discomfort or mess. Thus a user may sit, lie or move about without discomfort or embarrassment.

The reservoir 65 is at least partially sealed on an exterior face to contain the collection bag. The material used to seal the reservoir may be of any form of strap, netting, compressible foam or other material. The shape and material of the seat 60 is a matter of design choice. For example, it is envisioned that different shaped seats may be used in different situations; a lightweight and discreet seat may be used for those users who are generally mobile, but may not have control over their bowel movements. Such users may be able to quickly identify when the collection bag contains feces, and they can replace the collection bag before it becomes uncomfortable. Such a user may desire a seat that is relatively form fitted, so that it may be discreetly worn under clothes. Other users, such as those who lack mobility or the ability to communicate their discomfort, may benefit from a thicker seat, which provides more shielding to the user.

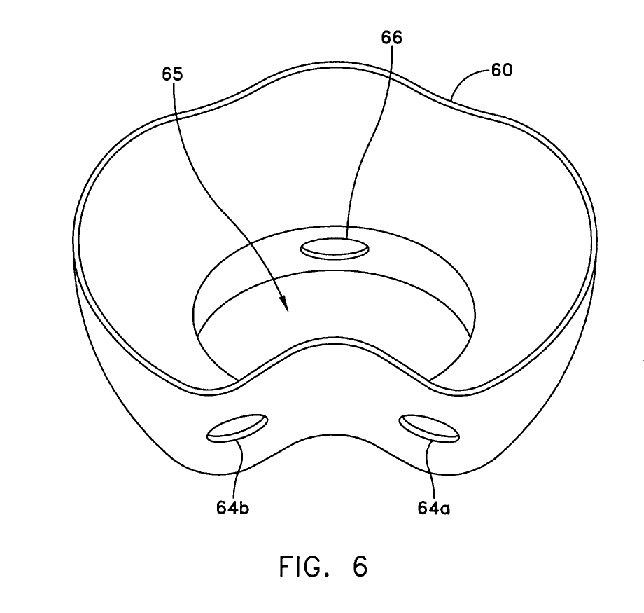

Referring now to FIG. 7, an illustration of the waste containment system with the coupled seat is shown. Note that the straps 24a, 24b and 26 slide through the pathways 64a, 64b, 66 of the seat 60, enabling the seat to the fixed to the system. The reservoir 65 accommodates the bag 56.

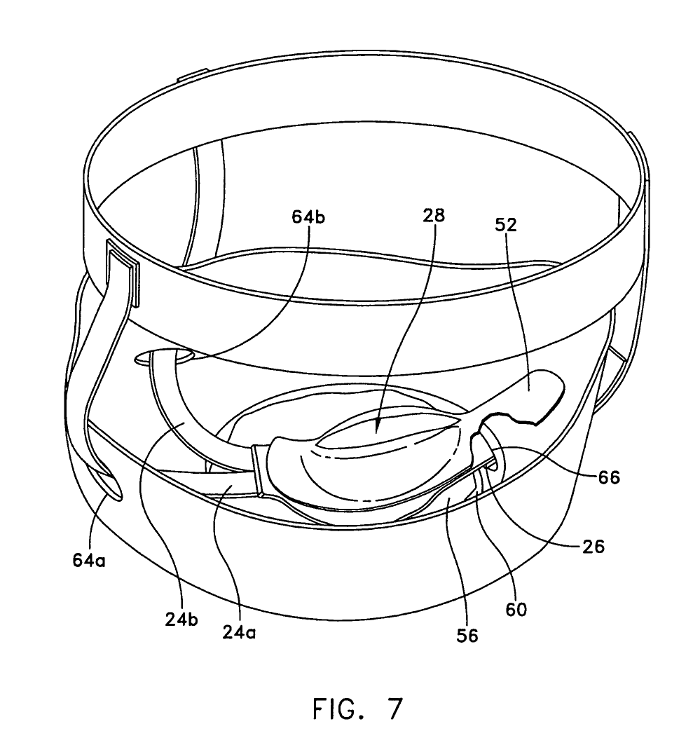

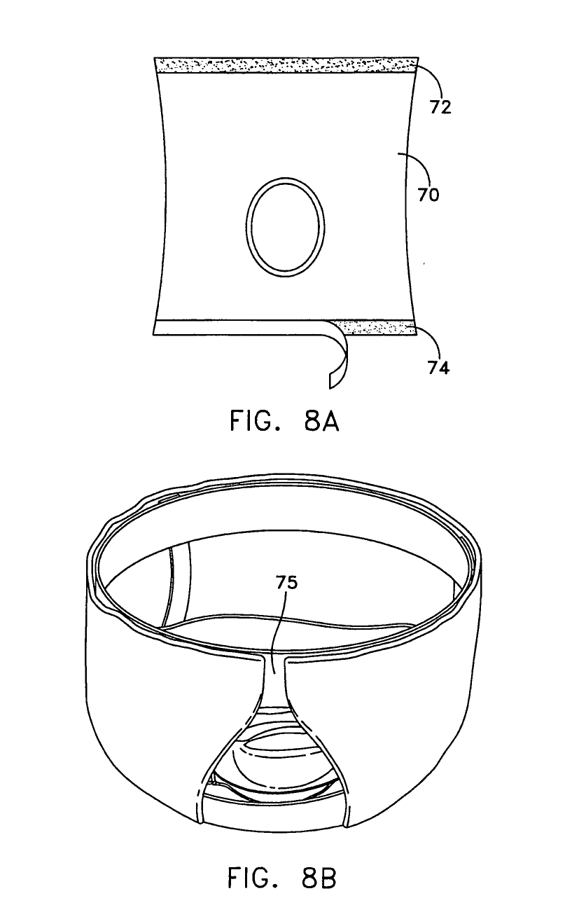

Accordingly, a waste containment system which may be used to contain fecal waste has been shown and described. It should be appreciated that the waste containment system of the present invention easily may be incorporated with other devices for capturing liquid waste. For example, as shown in FIGS. 8A and 8B, a diaper 70 advantageously may be modified to include adhesive means 72 and 74. The adhesive means may be hook and loop closures, such as Velcro, or other means for securing the diaper to the waist strap 15. The diaper may be fitted over the entire system as shown in FIG. 7b, with or without the molded seat 60.

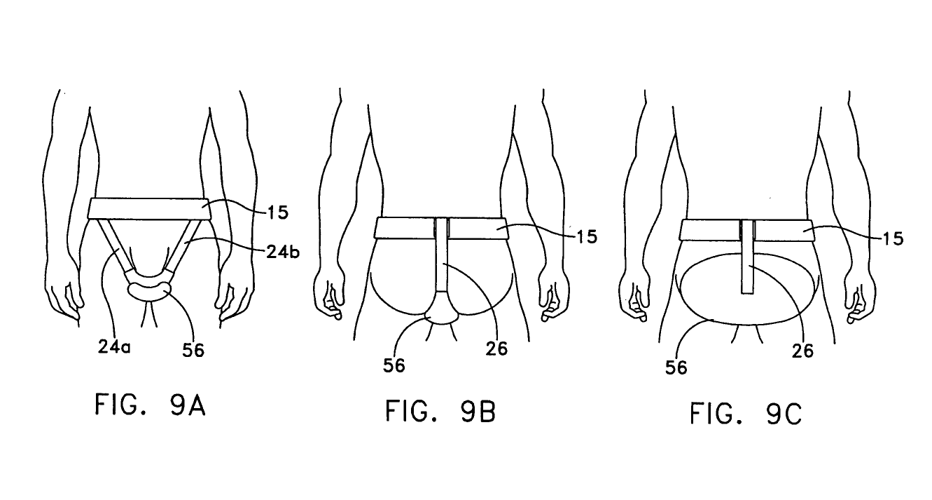

Referring now to FIGS. 9A-9C, several views of embodiments of the present invention as worn by an individual user are shown. FIG. 9A illustrates a front view of the waste collection device, including the waist strap 15, front straps 24a and 24b and the collection bag 56. In FIG. 9A, no seat is used. Such a configuration may be used, for example, by a person who is mobile but may be unable to dispose of the fecal waste when it occurs. Thus such a configuration may be helpful to those who are outdoors, hiking or in adverse conditions that do not include restroom facilities. The system permits such a user to defecate, than easily remove the collection bag. In such situations, the flap may advantageously be used to cleanse the anal area of waste. FIG. 9B is a back view of a user of the present invention, illustrating the collection bag and rear strap. FIG. 9C is also a back view of the present invention, with the seat integrated into the system

Accordingly a waste containment system having numerous advantages has been shown and described. The waste containment system includes a positionable orifice, removably adjusted to a waist strap to permit individual adjustment of the orifice position depending on user physical characteristics. A waste container includes a collection bag, made of a flexible non-allergenic material that is removably coupled to the orifice. With such an arrangement, the container may be easily replaced when it is full. The container advantageously includes a flap, which extends through the orifice and helps to secure the collection bag to the orifice, while cushioning the user from the container. A compressible seat with a reservoir may advantageously be coupled to the system to accommodate the collection bag when a user is sitting or lying down, minimize discomfort caused to the user in such situations. The system may easily be modified to incorporate existing waste disposal devices, such as diapers, if desired.

Having described exemplary embodiments, it will be appreciated that various modifications may be made without diverging from the spirit and scope of the invention, and that modification to and variation of the illustrated embodiments may be made without departing from the inventive concepts herein disclosed. Moreover, while the preferred embodiments are described in connection with various illustrative program command structures, one skilled in the art will recognize that the system may be embodied using a variety of specific command structures. Accordingly, the invention should not be viewed as limited except by the scope and spirit of the appended claims.

1. A waste containment system comprising:
a strapping system fixedly coupled to an orifice, for positioning the orifice in alignment with an anus of a user.

2. The waste containment system of claim 1, wherein the orifice is molded from elastomer.

3. The waste containment system of claim 1 wherein the orifice comprises synthetic rubber.

4. The waste containment system of claim 2, wherein the orifice comprises molded silicone.

5. The waste containment system of claim 1, wherein the orifice comprises molded polyurethane.

6. The waste containment system of claim 1 wherein a diameter of the orifice is selected in accordance with a diameter of an anus.

7. The waste containment system of claim 6, wherein the orifice is generally elliptically shaped.

8. The waste containment system of claim 1 further comprising a container, removably couplable to the orifice.

9. The waste containment system of claim 9 wherein the container comprises a collection bag and a neck, a first end of the neck is coupled to the collection bag, and a second end of the neck extends through the orifice.

10. The waste containment system of claim 9, further comprising a flap coupled to the second end of the neck, wherein the flap is matched in shape and form to the orifice.

11. The waste containment system of claim 10, further comprising a wipe coupled to the flap, wherein at least one face of the wipe is comprised of absorbent material.

12. The waste containment system of claim 9 wherein the collection bag is sized to accommodate storage of an average amount of fecal matter extruded in a 24 hour period.

13. The waste containment system of claim 9 wherein the collection bag is sized to accommodate at least 500 grams of fecal matter.

14. The waste containment system of claim 9 wherein the collection bag is generally rectangular in shape.

15. The waste containment system of claim 14 wherein the collection bag has a length in the range of 3-12 inches.

16. The waste containment system of claim 9 wherein the collection bag is comprised of an elastomer material.

17. The waste containment system of claim 16 wherein the elastomer material is synthetic rubber.

18. The waste containment system of claim 16 wherein the elastomer material is a nitrile material.

19. The waste containment system of claim 9 where in the neck ranges in length from 0.5 to 2 inches.

20. The waste containment system of claim 9 wherein the container further comprises a protrusion for affixing the container to the strapping system.

21. The waste containment system of claim 1 further including a waist strap, couplable to the strapping system, for securing the waste containment system on the user.

22. The waste containment system of claim 1 further including a seat, couplable to the strapping system, the seat including a reservoir for accommodating the container to permit the user to sit upon the container in comfort.

23. The waste containment system of claim 22, wherein the seat is comprised of compressible foam.

24. The waste containment system of claim 21 further including a diaper, couplable to the waist strap and enclosing the orifice and strapping system when in use.

25. A waste containment system comprising:
a waist strap;
a strapping system including orifice, the strapping system being couplable to the waist strap for aligning the orifice with the anus of a user; and
a container, removably couplable to the orifice and comprising a collection bag and a neck, wherein in use the neck of the container is extended through the orifice such that waste from the user is directed into the collection bag.

26. A waste containment system comprising:
means for directing fecal matter extruded by an individual into a collection bag; and
spacing means for accommodating the collection bag to reduce contact between the individual and the collection bag.

27. A waste container for use in a waste containment system comprising:
a collection bag;
a neck, coupled on a first end to the collection bag, the neck having a diameter sized in accordance with an orifice of a waste disposal system; and
a flap, coupled to a second end of the neck.
